(19) United States
(12) Patent Application Publication
(10) Pub. No.: US 2017/0202858 A1
(43) Pub. Date: Jul. 20, 2017

(54) COMBINATIONS OF TIOTROPIUM BROMIDE, FORMOTEROL AND BUDESONIDE FOR THE TREATMENT OF COPD

(71) Applicant: Teva Pharmaceuticals Europe B.V., Amsterdam (NL)

(72) Inventor: Michiel ULLMANN, Amsterdam (NL)

(73) Assignee: Teva Pharmaceuticals Europe B.V., Amsterdam (NL)

(21) Appl. No.: 15/310,133

(22) PCT Filed: May 8, 2015
(86) PCT No.: PCT/EP15/60256

§ 371 Date: Nov. 10, 2016

(30) Foreign Application Priority Data
May 12, 2014 (GB) 1408387.7

(51) Int. Cl. A61K 31/58; A61M 15/00; A61K 31/136; A61K 9/00; A61K 31/439

(52) U.S. Cl. CPC A61K 31/58; A61K 9/0075; A61K 31/439; A61K 31/136; A61M 15/003; A61M 15/009

(57) ABSTRACT

This invention provides a combination product comprising an inhalable long-acting muscarinic antagonist (LAMA) composition for use in the long-term treatment of COPD combined with an inhalable fixed-dose composition comprising budesonide and formoterol or a pharmaceutically acceptable salt thereof for administration pro re nata (prn) as a rescue medication for the treatment of acute exacerbations of COPD.

The present invention relates to the treatment of respiratory disorders, and particularly to a combination product/medicament for use in the treatment of chronic obstructive pulmonary disease (COPD).

COPD is a leading cause of death worldwide. Global trends indicate that case frequency will continue to rise and by 2030 COPD will become the fourth leading cause of death worldwide. COPD is considered a preventable and treatable disease and is characterised by persistent airflow limitation that is not fully reversible. The limitation is usually progressive, and primarily associated with an abnormal inflammatory response in the lungs to noxious particles or gases.

COPD is a heterogeneous long-term disease comprising chronic bronchitis, emphysema and also involving the small airways. The pathological changes occurring in patients with COPD are predominantly localised to the airways, lung parenchyma and pulmonary vasculature. Phenotypically, these changes reduce the healthy ability of the lungs to absorb and expel gases.

Bronchitis is characterised by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Their severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β₂-agonists and corticosteroids. The medicaments are often administered as a single therapy or as combination treatments.

Patients are categorised by the severity of their COPD using categories defined in the GOLD Guidelines (Global Initiative for Chronic Obstructive Lung Disease, Inc.). The categories are labelled A-D and the recommended first choice of treatment varies by category. Patient group A are recommended a short-acting muscarinic antagonist (SAMA) prn (pro re nata) or a short-acting β₂-aginist (SABA) prn. Patient group B are recommended a long-acting muscarinic antagonist (LAMA) or a long-acting β₂-aginist (LABA). Patient group C are recommended an inhaled corticosteroid (ICS)+a LABA, or a LAMA. Patient group D are recommended an ICS+a LABA and/or a LAMA.

Stable COPD may be indefinitely maintained, however the disease also manifests itself in an acute form, known in the art as an exacerbation. An exacerbation of COPD is an acute event characterised by a worsening of the patient's respiratory symptoms that is beyond the baseline day-to-day variations and can often lead to a change in medication. Exacerbations may be subcategorised as being mild, moderate or severe, based on, for example, required medications (e.g. oral corticosteroids) and outcomes (e.g. hospitalisation) but are effectively a spectrum of acute worsening of the disorder. Exacerbations can be precipitated by several factors, though it is widely accepted that common causes are respiratory tract infections (viral and bacterial), increased exposure to particulates (air pollution) and poor patient compliance (forgetting or resisting to take medication). These episodes negatively affect the patient's quality of life, accelerate the rate of decline of lung function and are often associated with significant mortality, particularly instances in which hospitalisation is required. During exacerbations patients that seek medical assistance are often treated with SABAs, ICSs and antibiotics, although recent findings have indicated that symptoms persist for several weeks following onset, which suggests that the underlying pathophysiology is not resolved by this approach. Furthermore, it is generally documented that COPD patients frequently experience changeable symptoms. As such, it is estimated that an alarming number of patients endure exacerbations, but choose not to report them, and as a direct result, they suffer irreparable lung damage. These findings highlight an unmet clinical need for improved therapies that manage both stable COPD and offer relief during an exacerbation.

Accordingly, the present invention provides a combination product comprising an inhalable long-acting muscarinic antagonist (LAMA) composition for use in the long-term treatment of COPD combined with an inhalable fixed-dose composition comprising budesonide and formoterol or a pharmaceutically acceptable salt thereof for administration pro re nata (prn) as a rescue medication for the treatment of acute exacerbations of COPD.

The present invention is based upon a combined treatment of maintenance dose of a LAMA, with budesonide (an ICS) and formoterol (a LABA) in a single device as a rescue therapy. This combination allows patients to receive the benefits of daily maintenance medication and rescue therapy, where the rescue therapy is contained within one prescribed dosage (termed a “fixed-dose combination” or “FDC”). Should the patient's symptoms deteriorate (upon experiencing an exacerbation) they will then use a rescue medication. Upon actuation of the device, the patient obtains a dose of formoterol that provides immediate additional bronchodilation and hence provides symptomatic relief, as well as providing an early add-on to the maintenance therapy by way of both the bronchodilating effect of the formoterol and the antiinflammatory effect of the budesonide. This approach serves to improve patient convenience and compliance through simplifying a multi-faceted treatment into two devices, where the two devices are in the form of a preventer and a reliever, which is well-understood by COPD patients.

The LAMA maintenance is typically provided for patient group B, although a LAMA is considered a useful alternative maintenance therapy for patient group A. The additional budesonide/formoterol is particularly important and beneficial in circumstances where the patient has started an episode of exacerbation, since it effectively steps the patient up to a higher level of therapy on a temporary basis and delays the point in the disease progression where a permanent step-up is required. The dose of budesonide helps to address inflammation that may underlie the worsening of symptoms and the formoterol provides further long-duration bronchodilation at a time when it is needed.

Thus, the present invention provides both for the long-term treatment of COPD and the treatment of acute exacerbations of COPD. The long-term treatment involves the administration of a maintenance dose every day. The treatment is typically over a period of more than 6 months, and usually more than 12 months. Many patients will receive the treatment palliatively. This aspect of the disease may be termed “stable COPD”. The acute treatment is for exacerbations, as defined hereinabove. Exacerbations are treated prn, that is, as required. The present invention improves patient care and maintains positive patient prognoses. It particularly provides a therapy that can offer daily symptomatic relief and reduces patient distress in the early stages of, and during, an exacerbation presenting in the home. For this reason, the budesonide/formoterol aspect of the therapy may be termed a “rescue medication”. It provides bronchodilation and combats persistent inflammation with directed treatment at the appropriate location in the lungs.

The LAMA may be tiotropium, aclidinium or glycopyrrolate (all preferably presented as the bromide salts), but is preferably tiotropium. Tiotropium is indicated as a maintenance bronchodilator to relieve symptoms of patients with COPD (or as an add-on maintenance bronchodilator treatment for asthma).

Tiotropium is (1α,2β,4β,7β)-7-[(hydroxidi-2-thienylacetyl)oxy]-9,9-dimethyl-3-oxa-9-azoniatricyclo-[3.3.1.0^{2,4}]nonane and is described in more detail in EP 0 418 716. Tiotropium, as the bromide salt, is marketed worldwide as Spiriva®. Spiriva® is available as a dry powder inhalation (DPI) formulation, or as an aqueous solution for use with the Respimat® soft-mist inhaler. The DPI formulation is formulated with lactose carrier and is contained in capsules, each containing 22.5 microgram tiotropium bromide monohydrate equivalent to 18 microgram tiotropium. The delivered dose is 10 microgram tiotropium.

The tiotropium bromide may be in the form of a tiotropium bromide solvate, a tiotropium bromide hydrate e.g. tiotropium bromide monohydrate, anhydrous tiotropium bromide or amorphous tiotropium bromide. In a preferred embodiment of the present invention, tiotropium bromide is presented in the form of solid amorphous particles comprising an intimate admixture of amorphous tiotropium bromide together with an amorphous saccharide, typically lactose as described in WO 2009/007687.

The amount of tiotropium will vary depending on the particular product, severity and patient. Typically, the amount of tiotropium (i.e. based on the weight of tiotropium without including contribution to the mass of the counterion) delivered per inhalation is 1-50 μg.

It is preferable that substantially all of the particles of the LAMA are less than 10 μm in size. This is to ensure that, when administered with a DPI, the particles are effectively entrained in the air stream and deposited in the lower lung, which is the site of action. Preferably, the particle size distribution of the LAMA is d10<1 μm, d50=<5 μm, d90=<10 μm and NLT 99%<10 μm.

Budesonide is an inhaled corticosteroid. It is preferable that substantially all of the particles of the corticosteroid are less than 10 μm in size. This is to ensure that, when administered with a DPI, the particles are effectively entrained in the air stream and deposited in the lower lung, which is the site of action. Preferably, the particle size distribution of the corticosteroid is d10<1 μm, d50=<5 μm, d90=<10 μm and NLT 99%<10 μm.

The delivered dose of budesonide (the amount actually delivered to the patient) is preferably 50-500 μg per actuation, with specific examples being 80, 160 and 320 μg per actuation. Again, the actual prescribed dosage will be dependent upon patient age and weight, severity of disease and response to therapy.

Formoterol is a long-acting β₂-agonist that displays a rapid onset of action. It can be synthesised as four independent stereoisomers, and the present invention can include each of these individual forms. Typically it is administered as (R,R)-formoterol, or a racemic mixture of (R,R)- and (S,S)-formoterol. Suitable pharmaceutically acceptable salts of formoterol include those known in the art, and they are commonly derived from the addition of inorganic or organic acids to the medicament. Non-exhaustive examples include hydrochloride, hydrobromide, acetate, formate, halo and alkyl benzoate, tartrate, citrate, fumarate, triflate or salicylate. An example of particular interest is formoterol fumarate, e.g. formoterol fumarate dihydrate.

It is preferable that substantially all of the particles of formoterol fumarate are less than 10 μm in size. This is also to ensure that the particles are effectively entrained in the air stream and deposited in the lower lung, which is the site of action. Preferably, the particle size distribution of the formoterol is d10<1 μm, d50=<5 μm, d90=<10 μm and NLT 99%<10 μm; more preferably, the particle size distribution of the formoterol fumarate is d10<1 μm, d50=1-3 μm, d90=3.5-6 μm and NLT 99%<10 μm.

The delivered dose of formoterol, is preferably 1-20 μg per actuation, with specific examples being 4.5 and 9 μg per actuation. The doses are based on the amount formoterol present (i.e. the amount is calculated without including contribution to the mass of the counterion, where present). The actual prescribed dosage will be dependent upon patient age and weight, severity of disease and response to therapy.

Particularly preferred delivered doses of budesonide/formoterol in μg are 80/4.5, 160/4.5 and 320/9. Particularly preferred molar ratios of budesonide/formoterol are within the range of 40:1 to 10:1, wherein the moles of formoterol are based on the amount present (i.e. the amount is calculated without including contribution to the mass of the counterion).

The formulations may be administered via inhalation devices known in the art. These can include but are not limited to dry powder inhalers (DPIs) and pressurised metered dose inhalers (pMDls). DPIs are preferred for both inhalers.

The compositions are preferably dry powder formulations, further comprising a coarse carrier. The carrier can be selected from saccharides e.g. glucose or lactose. The carrier is preferably lactose, more preferably lactose monohydrate (α-lactose monohydrate) and may be prepared by standard techniques, e.g. sieving. The lactose carrier preferably has a particle size distribution of d10=20-65 μm, d50=80-120 μm, d90=130-180 μm and <10 μm=<10%. Preferably, the particle size distribution of the lactose is d10=20-65 μm, d50=80-120 μm, d90=130-180 μm and <10 μm=<6%.

A suitable inhaler for working the present invention is the Spiromax® DPI available from Teva Pharmaceuticals, see WO 92/10229 and WO 2011/054527.

The delivered dose of the active agent is measured as per the USP <601>, using the following method. A vacuum pump (MSP HCP-5) is connected to a regulator (Copley TPK 2000), which is used for adjusting the required drop pressure P₁ in a DUSA sampling tube (Dosage Unit Sampling Apparatus, Copley). The inhaler is inserted into a mouthpiece adaptor, ensuring an airtight seal. P₁ is adjusted to a pressure drop of 4.0 KPa (3.95-4.04 KPa) for the purposes of sample testing. After actuation of the inhaler, the DUSA is removed and the filter paper pushed inside with the help of a transfer pipette. Using a known amount of solvent (acetonitrile:methanol:water (40:40:20)), the mouthpiece adaptor is rinsed into the DUSA. The DUSA is shaken to dissolve fully the sample. A portion of the sample solution is transferred into a 5 mL syringe fitted with Acrodisc PSF 0.45 μm filter. The first few drops from the filter are discarded and the filtered solution is transferred into a UPLC vial. A standard UPLC technique is then used to determine the amount of active agent delivered into the DUSA. The delivered doses of the inhaler are collected at the beginning, middle and end of inhaler life, typically on three different days.

In one embodiment of the LAMA composition is administered 2-4 times per day as a maintenance dose, more preferably the composition is administered twice-per-day (i.e. bid) as a maintenance dose. Bid administration is typically every morning and every evening as a maintenance dose, and the required dose may be administered in one or two puffs of the inhaler.

The budesonide/formoterol composition is preferably administered no more than ten times prn as a rescue medication, more preferably no more than eight times prn as a rescue medication. In a particularly preferred embodiment, the LAMA composition is administered twice-per-day as a maintenance dose and the budesonide/formoterol composition no more than eight times prn as a rescue medication. Ideally, the patient should not exceed 120 μg of formoterol over any 24 hour period and 3,200 μg of budesonide over any 24 hour period.

Typically, the fixed-dose budesonide/formoterol composition is used solely for rescue use. However, in a further embodiment of the present invention, the inhalable fixed-dose composition comprising budesonide and formoterol or a pharmaceutically acceptable salt thereof may additionally be used as part of the maintenance therapy alongside the LAMA, and alongside its use as a rescue medicine. In this embodiment, the patient is provided with a maintenance therapy of both the LAMA composition and the fixed-dose budesonide/formoterol composition, and then uses the fixed-dose budesonide/formoterol composition additionally for rescue use.

Accordingly, the present invention also provides a combination product comprising an inhalable long-acting muscarinic antagonist (LAMA) composition combined with an inhalable fixed-dose composition comprising budesonide and formoterol or a pharmaceutically acceptable salt thereof, wherein the LAMA composition is used for the long-term treatment of COPD, and the fixed-dose composition of budesonide and formoterol is used the long-term treatment of COPD and for administration pro re nata (prn) as a rescue medication for the treatment of acute exacerbations of COPD.

In this embodiment, the fixed-dose budesonide/formoterol composition is administered 2-4 times per day as a maintenance dose, more preferably the composition is administered twice-per-day (i.e. bid) as a maintenance dose. Bid administration is typically every morning and every evening as a maintenance dose, and the required dose may be administered in one or two puffs of the inhaler. The maintenance dose is preferably provided at the same time as the LAMA composition, principally to aid patient compliance. This maintenance dosage regimen is the recommended first choice for patients in group D, i.e. with the most severe form of COPD.

A beneficial feature of this approach is that the patient not only experiences relief from receiving a β₂-agonist but also receives an additional dose of steroid. This feature of the invention is particularly beneficial in circumstances where the patient has missed a maintenance dose of the budesonide/formoterol composition.

The restrictions on the rescue use are the same as where the fixed-dose budesonide/formoterol composition is used solely for rescue use. That is, the budesonide/formoterol composition is preferably administered no more than ten times prn as a rescue medication, more preferably no more than eight times prn as a rescue medication. Ideally, the patient should not exceed 120 μg of formoterol over any 24 hour period and 3,200 μg of budesonide over any 24 hour period.

The combination product of the present invention is preferably provided as a first and second inhaler. In this embodiment, the product comprises a first inhaler containing the LAMA composition and a second inhaler containing the fixed-dose composition of budesonide and formoterol. The present invention also provides a kit containing a first inhaler containing an inhalable long-acting muscarinic antagonist (LAMA) composition, a second inhaler containing an inhalable fixed-dose composition comprising budesonide and formoterol or a pharmaceutically acceptable salt thereof, and optionally instructions for use. The first inhaler is for use in the long-term treatment of COPD combined and the second inhaler is for administration pro re nata (prn) as a rescue medication for the treatment of acute exacerbations of COPD. Embodiments and preferred features of the combination product discussed herein apply equally to the kit.

The present invention further provides a combination therapy for treating COPD comprising administering an inhalable long-acting muscarinic antagonist (LAMA) composition for the long-term treatment of COPD combined with administering an inhalable fixed-dose composition comprising budesonide and formoterol or a pharmaceutically acceptable salt thereof for administration pro re nata (prn) as a rescue medication for the treatment of acute exacerbations of COPD. Embodiments and preferred features of the combination product discussed herein apply equally to this method.

The present invention will now be described with reference to the examples, which are not intended to be limiting.

### EXAMPLES

### Example 1

Three formulations of Budesonide/Formoterol (BF) Spiromax (Teva Pharmaceuticals) were prepared: low strength (120 inhalations, each delivering 80 μg budesonide and 4.5 μg formoterol), middle strength (120 inhalations, 160 μg budesonide and 4.5 μg formoterol per inhalation), and high strength (60 inhalations, 320 μg budesonide and 9 μg formoterol per inhalation).

The compositions of the three strengths of BF Spiromax per container are set out in Tables 1-3.

TABLE 1
Composition per container of BF Spiromax
80/4.5 μg 120 inhalation product

| Material | Weight | | Function | Quality Standard |
|---|---|---|---|---|
| Budesonide (micronised) | 12.0 | mg | Drug substance | Ph. Eur. |
| Formoterol fumarate | 0.645 | mg | Drug substance | Ph. Eur. |
| dihydrate (micronised) | | | | |
| Lactose monohydrate | 1.487 | g | Excipient | Ph. Eur. |
| Target fill weight per | 1.500 | g | | |
| device | | | | |

TABLE 2
Composition per Container of BF Spiromax
160/4.5 μg 120 inhalation product

| Material | Weight | | Function | Quality Standard |
|---|---|---|---|---|
| Budesonide (micronised) | 31.6 | mg | Drug substance | Ph. Eur. |
| Formoterol fumarate | 0.914 | mg | Drug substance | Ph. Eur. |
| dihydrate (micronised) | | | | |
| Lactose monohydrate | 0.838 | g | Excipient | Ph. Eur. |
| Target fill weight per | 0.870 | g | | |
| device | | | | |

TABLE 3
Composition per Container of BF Spiromax
320/9 μg 60 inhalation product

| Material | Weight | | Function | Quality Standard |
|---|---|---|---|---|
| Budesonide (micronised) | 28.7 | mg | Drug substance | Ph. Eur. |
| Formoterol fumarate | 0.870 | mg | Drug substance | Ph. Eur. |
| dihydrate (micronised) | | | | |
| Lactose monohydrate | 0.840 | g | Excipient | Ph. Eur. |
| Target fill weight per | 0.870 | g | | |
| device | | | | |

Tiotropium bromide formulation is prepared according to the examples of WO 2009/007687. The matrix is prepared by combining a solution of tiotropium bromide in water with a solution of lactose in water, and spray drying the resultant solution. This spray drying process results in a 5% w/w tiotropium in lactose spray dried matrix. The mass median diameter (MMD, or D50) of the matrix particles is approximately 2 μm. The matrix is composed of amorphous tiotropium bromide and amorphous lactose. A coarse lactose carrier, Respitose SV003 (DMV) or InhaLac 230 (Meggle), is added to the matrix to provide the inhalable formulation.

### Example 2

This is a two-arm parallel study investigating whether symptom-driven maintenance and reliever/rescue therapy with tiotropium bromide and budesonide/formoterol is more effective as a treatment regimen that manages and also concomitantly reduces the number of exacerbations of COPD compared to a multiple device fixed maintenance dose of fluticasone/salmeterol and salbutamol as a rescue medication.

### Patient Group A (Invention)

Participants are receiving Spiromax® tiotropium bromide, two inhalations, twice daily and additionally, Spiromax® budesonide/formoterol 160/4.5 μg as needed, with a maximum of eight additional inhalations per day for rescue use.

### Patient Group B (Comparative)

Participants are receiving Diskus® fluticasone/salmeterol (steroid/long-acting β₂-agonist) 500/50 μg, one inhalation, twice daily and additionally, salbutamol (short-acting β₂-agonist) 100 μg as needed with a maximum of eight additional inhalations per day. The comparative study represents an example of the current standard treatment for COPD.

Patients are being subjected to constant evaluation throughout the investigation. Key parameters that are being assessed include; but are not limited to, reduction in the number of exacerbations (moderately severe and severe exacerbations combined), reductions in hospitalisation during exacerbations, improvement in patient compliance and convenience, general lung function (PEF, FEV₁, FEV₁/FVC, FEV25-75%, RV, TLC, RV/TLC, RV/TLC %, predicted).

1. (canceled)

2. (canceled)

3. (canceled)

4. (canceled)

5. (canceled)

6. (canceled)

7. (canceled)

8. (canceled)

9. A kit comprising a first inhaler containing an inhalable long-acting muscarinic antagonist (LAMA) composition, a second inhaler containing an inhalable fixed-dose composition comprising budesonide and formoterol or a pharmaceutically acceptable salt thereof, and optionally instructions for use.

10. The kit of claim 9, wherein both compositions are dry powder formulations, each further comprising a coarse carrier.

11. The kit of claim 9, wherein the LAMA is tiotropium.

12. The kit of claim 11, wherein the first inhaler provides a delivered dose of tiotropium, based on the amount of tiotropium, of 1-50 μg.

13. The kit of claim 9, wherein the second inhaler provides a delivered dose of formoterol, based on the amount of formoterol, of 1-20 μg and delivered dose of budesonide of 5-500 μg.

14. The kit of claim 9, wherein the LAMA composition is for use in the long-term treatment of COPD and the fixed-dose composition of budesonide and formoterol is for administration pro re nata (prn) as a rescue medication for the treatment of acute exacerbations of COPD.

15. The kit of claim 14, wherein the LAMA composition is used for the long-term treatment of COPD, and the fixed-dose composition of budesonide and formoterol is used for the long term treatment of COPD and for administration pro re nata (prn) as a rescue medication for the treatment of acute exacerbations of COPD.

16. A method for the long-term treatment of COPD and for the treatment of acute exacerbations of COPD in a patient comprising
administering to the patient a maintenance dose of an inhalable long-acting muscarinic antagonist (LAMA) composition for the long-term treatment of COPD; and
administering to the patient an inhalable fixed-dose composition comprising budesonide and formoterol, or a pharmaceutically acceptable salt thereof, pro re nata (prn), for the treatment of the acute exacerbations of COPD.

17. The method of claim 16 wherein administering to the patient a maintenance dose comprises administering the inhalable long-acting muscarinic antagonist (LAMA) composition 2-4 times per day.

18. The method of claim 16, wherein both compositions are dry powder formulations, each further comprising a coarse carrier.

19. The method of claim 16, wherein the LAMA composition is administered from a first inhaler, and the fixed-dose composition is administered from a second inhaler.

20. The method of claim 16, wherein the delivered dose of the formoterol, or a pharmaceutically acceptable salt thereof, is 1-20 μg, based on the amount of formoterol.

21. The method of claim 16, wherein the delivered dose of the budesonide is 5-500 μg.

22. The method of claim 16, wherein the LAMA is tiotropium, or a bromide salt thereof.

23. The method of claim 22, wherein the delivered dose of the tiotropium, or a bromide salt thereof, is 1-50 μg, based on the amount of tiotropium.

24. The method of claim 16, further comprising administering to the patient a maintenance dose of the fixed-dose composition of budesonide and formoterol, or a pharmaceutically acceptable salt thereof, for the long-term treatment of COPD.
